SEQUENCE LISTING (8 sequences)

SEQ ID NO: 1 - protein, 12 aa, Homo sapiens
  MISC_FEATURE (1)..(1): Xaa=pyroglutamic acid
XGPWLERPPPPC

SEQ ID NO: 2 - protein, 11 aa, Homo sapiens
  MISC_FEATURE (1)..(1): Xaa=pyroglutamic acid
XGPWLRPPPPC

SEQ ID NO: 3 - protein, 9 aa, Homo sapiens
  MISC_FEATURE (1)..(1): Xaa=pyroglutamic acid
XGWRPPPPC

SEQ ID NO: 4 - protein, 16 aa, Homo sapiens
  MISC_FEATURE (1)..(1): Xaa=pyroglutamic acid
XGPWLEEEESSPPPPC

SEQ ID NO: 5 - protein, 6 aa, Artificial Sequence
  Hypothetical spacer peptide
RPPPPC

SEQ ID NO: 6 - protein, 7 aa, Artificial Sequence
  hypothetical spacer peptide
SSPPPPC

SEQ ID NO: 7 - protein, 17 aa, Homo sapiens
KLNRSVQGTGPGPGASL

SEQ ID NO: 8 - protein, 15 aa, Homo sapiens
GPGAHRALSGAPISF

(19) United States
(12) Patent Application Publication
(10) Pub. No.: US 2011/0117108 A1
(43) Pub. Date: May 19, 2011

(54) TREATMENT AND PREVENTION OF CANCEROUS AND PRE-CANCEROUS CONDITIONS OF THE LIVER, LUNG AND ESOPHAGUS

(75) Inventors: Philip C. Gevas, Key Biscayne, FL (US); Dov Michaeli, Key Biscayne, FL (US); Stephen Grimes, Key Biscayne, FL (US)

(73) Assignee: Cancer Advances, Inc., Durham, NC (US)

(21) Appl. No.: 13/012,433

(22) Filed: Jan. 24, 2011

Related U.S. Application Data
(63) Continuation of application No. 10/192,257, filed on Jul. 9, 2002, now abandoned.
(60) Provisional application No. 60/303,868, filed on Jul. 9, 2001.

(51) Int. Cl. A61K 39/395; A61K 39/00; A61P 35/00

(52) U.S. Cl. 424/158.1; 424/185.1; 424/184.1

(57) ABSTRACT

The invention relates to the treatment and/or prevention of cancerous and/or, precancerous conditions of the liver, lung and esophagus by actively and/or passively immunizing a patient against the peptide hormone gastrin and/or a gastrin receptor, e.g., the CCK-B/gastrin receptor. The immunizations of the invention may be employed as a monotherapy, an adjunctive therapy, or as part of a combination therapy.

### RELATED APPLICATIONS

The present U.S. patent application is a continuation of U.S. patent application Ser. No. 10/192,257, filed Jul. 9, 2002, which claims the benefit of U.S. Provisional Patent Application Ser. No. 60/303,868, filed Jul. 9, 2001. The disclosure of each of these applications is incorporated herein by reference in its entirety.

### FIELD OF THE INVENTION

The present invention relates to methods and compositions for the treatment and prevention of cancerous and pre-cancerous conditions of the liver, lung and esophagus. The invention also relates to the prevention and/or inhibition of metastasis of a gastrin-induced malignancy to a site in the liver, lung or esophagus.

### BACKGROUND OF THE INVENTION

Gastrin is a growth factor that has been shown to promote the growth of normal gastrointestinal mucosa as well as a variety of cancers including gastric, colonic, rectal, pancreatic, hepatocellular and neuronal malignancies. In particular, gastrin is now a well recognized growth factor for certain human tumors, e.g., gastrinomas and colorectal adenocarcinomas, including metastases (see Watson et al. 2000 for a review, Smith et al. 1989, Seitz et al. 1991 and Wong et al. 1991). (The full citations of the references cited herein, where not recited in the text, are provided in the Reference Section preceding the Claims). Elevated plasma levels of total gastrin occurs in patients with colorectal cancers, and in particular, increased amounts of the hormone precursor, progastrin, have been detected in many colorectal tumors using gastrin antisera (Ciccotosto et al. 1995). As used here, the term “colorectai” is a subset of gastrointestinal.

The increased gastrin level in colorectal tumors is, in part, attributed to the aberrant expression of the gastrin gene in the colorectal tumor cells (Hoosein et al. 1990, Baldwin et al. 1992 and Finley et al. 1993). Gastrin-like peptides have been identified in such cells (Hoosein et al. 1988, Watson et al. 1991 and Finley et al. 1993), and were confirmed to be precursor gastrin species (Van-Solinge et al. 1993 and Nemeth et al. 1993).

Serum-associated G17 has the potential to stimulate the growth of colorectal tumors in an endocrine manner mediated by CCK-B/gastrin receptors (Watson et al. 1993). Gastrin-17 appears to be particularly implicated in stimulating the growth of human colorectal adenocarcinomas due to a possible increased affinity for gastrin/cholecystokinin (CCK)-B receptors on the tumor cells, over other gastrin hormone species (Rehfeld. J. F. 1972). The CCKB/gastrin receptors were found to be expressed in a high affinity form on 56.7% of human primary colorectal tumors (Upp et al. 1989). It has been postulated that a potential autocrine loop may also exist due to endogenous production of precursor gastrin peptides by such tumors (Van-Solinge et al. 1993 and Nemeth et al. 1993), as it has recently been shown that the precursor gastrin molecule, glycine-extended gastrin 17 (G17-Gly), stimulated the growth of a gastrointestinal tumor cell line. The trophic effects of G17-Gly on tumors has been shown to be mediated by a receptor other than the CCK-B/gastrin receptor and an autocrine growth loop, possibly involving gastrin precursors, has been postulated to be involved in the proliferation of gastrointestinal tumors (Seva et al. 1994).

Surgery is the most effective method for treating operable colon cancers. Resection of the primary tumors in the colorectal area, for example, does not always remove all malignant tissue, since undetectable “occult” or “micrometastases” may exist. In addition, during the physical action of cutting the primary tumors, tumor cells may break off and travel through the circulation, establishing themselves in the liver or other sites in the body. Colorectal adenocarcinomas most commonly metastasize in the liver.

Surgical treatment of liver metastases in patients with colorectal cancer leads to complications. Since the liver can regenerate, liver resection promotes the release of a number of trophic agents which are thought to contribute to liver regeneration (Leith et al. 1992, Mizutani et al. 1992, Ballantyne et al. 1993, Vaillant et al. 1993, Ledda-Columbano et al. 1993, Matsumata et al. 1995, Slooter et al. 1995, Hananel et al. 1995) including, insulin, glucagon, somatostatin (Junge et al. 1977), fibroblast growth factor (FGF), epidermal growth factor, (EGF) (Gutman et al. 1994-95), transforming growth factor a (TGFa), interleukin-6, hepatocyte growth factor, and tumor necrosis factor (de Jong et al. 1996).

Gastrin 17 has also been found to have a trophic effect on normal and regenerating liver cells and on liver cells after injury, such as with alcohol damage or liver surgery. Two- to five-fold increases in gastrin levels have been recorded after liver injury, with maximal gastrin levels found at 24-72 hours after injury. The high levels of gastrin are thought to be required to stimulate or induce the hepatic cells to proliferate, since liver tissue can regenerate after injury. Gastrin levels gradually decrease to normal beginning at 72 hours after liver injury. Gastrin is also required for the proper establishment of metastatic colorectal carcinoma cells in the liver. In addition, cells from primary liver cancer or hepatocellular carcinoma, commonly known as “hepatoma” have gastrin receptors and thus proliferate in response to gastrin. Most liver tumors express the CCK-B/gastrin receptor and precursor forms of gastrin (Caplin 1999).

Although surgery is the most effective method for treating colorectal tumors, hepatomas and metastatic tumors in the liver (Supe et al. 1994, Fong et al. 1993, de Jong et al. 1996, Vauthey et al. 1995, Scheele et al. 1991, Ballantyne et al. 1993, Katoh et al. 1990), approximately 90% of the patients with these tumors in the liver cannot be surgically treated because in many instances the tumors cannot be located or are present in anatomic sites that are inoperable. These patients die within one year of their tumors being diagnosed. For the remaining 10% of the patients with liver colorectal liver metastases or hepatomas that have resectable tumors, it has been reported that approximately 50% are cured since no recurrence of tumors has been observed (Goletti et al. 1992 and Katoh et al. 1990). However, clinical data indicate that even though the life-span of the patient is prolonged with surgery for the remaining 50% of patients with resectable tumors, all will have recurrence of the tumors 2 years after the surgery, and 5 years after surgery 70% of the patients will have tumor regrowth. Patients with tumor regrowth have 50% of the tumors within the liver and 50% in other places in the body, such as the lung, bowel and peritoneum (Scheele et al. 1991, Vauthey et al. 1995, Ballantyne et al., 1993). Thus, hepatic resection is presently the most effective therapy for the treatment of hepatomas and liver colorectal metastases.

Present standard therapies after liver resection include treatments with chemotherapeutic agents, such as 5-fluorouracil, leucovorin, cisplatin, tumor necrosis alpha factor (Fong et al., 1995) and proglumide, a gastrin antagonist (Kameyama et al. 1994). In most instances, these tumors do not respond well to radiation or chemotherapy regimens, and new treatments are needed to supplement present procedures. For tumors that are operable, it is not known if all malignant tissue is removed or if metastatic cells have broken off from the tumor prior to or during surgery, or if micrometastases are present in the patient which are capable of tumor regrowth somewhere else in the body.

For gastrin-dependent tumors, a number of high affinity CCK-B/gastrin receptor antagonists have been described, such as L-365,260 (Bock et al. 1989) and CI-988 (Hughes et al. 1990), which have been shown to effectively neutralize the effects of exogenous gastrin on gastrin-dependent tumor growth both in vitro and in vivo (Watson et al., Kameyana et al. and Romani et al. 1994). However, the antagonists lack specificity as they block the actions of all the potential ligands of the receptor, such as gastrin-34 (G34) and CCK. Moreover, the cellular receptors which recognize and bind the gastrin precursor, G17-Gly, do not bind all the inhibitors tested (Seva et al. 1994). Thus, if a distinct receptor is involved in the autocrine growth cascade, then the gastrin antagonists may be unable to block this mechanism of tumor growth promotion. Radiolabeled gastrin- and cholecystokinin-related peptides have also been investigated for use as in vivo targeting agents for CCK-B/gastrin receptor expressing tumors. See Behr et al., Cholecystokinin-8/gastrin receptor binding peptides: preclinical development and evaluation of their diagnostic and therapeutic potential, Clin Cancer Res (1999) October: 5 (10 Suppl): 3124s-3138s, which is hereby incorporated by reference.

A therapeutic method of selectively immunologically neutralizing the biological activity of the gastrin 17 hormone both in mature and glycine-extended precursor forms would provide an effective means of controlling or preventing gastrin-dependent tumor regrowth resulting from excessive gastrin 17 hormone production.

Co-assigned U.S. Pat. Nos. 5,023,077 and 5,468,494, which are hereby incorporated by reference, disclose immunogenic compositions useful for controlling G17 and G34 levels in a patient by generating anti-gastrin antibodies, and the use of such compositions for the treatment of gastric and duodenal ulcers and gastrin-induced cancers. The present invention also concerns the use of the anti-G17 immunogenic compositions disclosed in the U.S. Pat. Nos. 5,023,077 and 5,468,494 in the prevention of tumor regrowth and/or the development of metastatic cancers after liver resection, wherein the regrowth of the tumors is stimulated by gastrin 17, since tumor recurrence after surgery is a common problem, particularly, after liver resection. The present invention also concerns immunization against the CCKB/gastrin receptor to block activation of receptor on the tumor cells. Co-assigned U.S. application Ser. No. 09/076,372 discloses methods for the preparation of immunogens eliciting an antibody response to the CCK-B/gastrin receptor, and is hereby incorporated by reference.

The method of the present invention for preventing metastatic tumor growth or tumor regrowth as a cancer therapy described has several advantages over present treatment methods. The method is non-invasive, selectively reversible, does not damage normal tissue, does not require frequent repeated treatments, and does not cross the blood brain barrier.

Gastrin is associated with lung cancer arising in the lung. See Gocyk et al., 2000 which is hereby incorporated by reference. Similarly to the above-discussed liver cancer, gastrin is also associated with lung cancer metastasized from gastrointestinal malignancies. The present invention relates to the treatment of lung cancers and to the prevention of metastasis to the lung, by blocking the gastrin-dependent activation of the CCK-B/gastrin receptor expressed on tumor cells. Moreover, the present invention is directed to the treatment of both small cell lung cancer (SCLC) and non-small cell lung cancer (NSCLC). Various types of treatment regimens continue to be developed for SCLC and NSCLC. See Reddy, 2000 for SCLC and Evans, 2001 for NSCLC, which articles are hereby incorporated by reference.

Barrett's esophagus is a pre-cancerous condition arising in 10-20% of gastroesophageal reflux disease (GERD) sufferers. Approximately, 20 Million U.S. citizens are afflicted with GERD. Approximately 5-10% of Barrett's esophagus cases will progress to the cancerous state, specifically adenocarcinoma. (See National Institutes of Health publication No. 99-4546, May 1999) Current preventative therapies and therapeutic treatments are reviewed in Fennerty, 2001, which is hereby incorporated by reference. Various studies have suggested the presence of gastrin and/or gastrin secreting cells in Barrett's esophagus lesions and, therefore, a role for gastrin in promoting the Barrett's esophagus lesion and its progression to a cancerous state, i.e., adenocarcinoma, is suggested. See, e.g., Buchanan et al. Regulatory peptides in Barrett's oesophagus, J. Pathol (1985) July; 146(3): 227-34 and Trakal et al., Diagnosis and etiology of Barrett's esophagus: Presence of gastrin secreting cells, Acta Gastroenterol Latinoam (1985); 15(2): 67-80, which articles are hereby incorporated by reference. The present invention relates to the treatment of Barrett's esophagus and the prevention or delay of the progression of Barrett's esophagus to esophageal adenocarcinoma. The invention also relates to the treatment of pre-existing esophageal adenocarcinomas and other malignancies of the esophagus.

### SUMMARY OF THE INVENTION

The invention relates to the treatment and/or prevention of cancerous and/or precancerous conditions of the liver, lung and esophagus by actively and/or passively immunizing a patient against the peptide hormone gastrin and/or a gastrin receptor, e.g., the CCK-B/gastrin receptor. The immunizations of the invention may be employed as a monotherapy, an adjunctive therapy, or as part of a combination therapy with, e.g. chemotherapy and/or radiotherapy agents.

The invention provides compositions and methods for inhibiting metastasis of gastrin promoted tumor cells to the liver, lung and esophagus from, e.g., a gastrointestinal malignancy. The invention also provides compositions and methods for treating gastrin-promoted malignancies of the liver, lung and esophagus. The invention provides compositions and methods for treating both small cell lung cancers and non-small cell lung cancers. The invention also provides a combined therapy for the treatment of non-small cell lung cancer which comprises active and/or passive immunization against gastrin and/or its receptor, in combination with administration of a taxane, such as docetaxel. The invention further provides compositions and methods for inhibiting the transition of pre-malignant (pre-cancerous) cells of the liver, lung or esophagus to a cancerous state.

### DETAILED DESCRIPTION OF THE INVENTION

The methods comprise the active or passive immunization of a patient with anti-G17 immunogen or antibodies against gastrin 17 hormone in order to control the patient's gastrin 17 levels in order to treat or prevent the progression of cancerous and/or pre-cancerous conditions of the lung, liver or esophagus. The invention also relates to preventing the successful metastasis of gastrin-dependent tumor cells to liver, lung and esophagus. U.S. Pat. Nos. 5,023,077 and 5,785,970 disclose methods of actively and passively immunizing patients against gastrin and are hereby incorporated by reference.

The immunization of the invention may be employed as a monotherapy, an adjunctive therapy to surgery, chemotherapy and/or radiotherapy, or as part of a combination therapy comprising, e.g., chemotherapy agents, radiotherapy agents, biological agents such as modified viruses, and/or photodynamic therapy treatments.

By inducing anti-gastrin 17 antibodies in a patient, the hormone gastrin 17 and the prohormone progastrin G17-Gly are neutralized in vivo, so as to inhibit their physiological effects. In particular, the neutralization of G17 prevents the binding of the hormone to its physiological receptors, thereby inhibiting the growth of the tumor cells.

The anti-G17 immunogens, comprise immunomimic fragments of the N-terminal amino acids of G17 conjugated to an immunogenic carrier such as Diphtheria toxoid (DT), by a spacer peptide, and raise antibodies which bind and neutralize G17.

In one embodiment of the invention, the method of immunization against G17 comprises active immunization, wherein a patient is immunized with an immunogen of the invention. The immunogen stimulates the production of antibodies against G17 in the immunized patient, inducing sufficient antibody titers to neutralize and inhibit the physiological effects of G17 so as to limit the cancer-trophic hormone levels produced by the patient's liver cells in response to the surgery. The physiological neutralization of the G17 hormone by the anti-G17 antibodies produced in the patient inhibits gastrin, thereby preventing the regrowth of tumor cells which dependent on G17 as the growth stimulator or inducer. The treatment methods of the invention are particularly suited for the treatment of G17-responsive gastrin-dependent metastatic tumor cells after liver resection.

The immunogens of the invention comprise peptides composed of two functional regions: an immunomimic region and a spacer region. The function of the immunomimic region which immunologically cross-reacts with G17 is to induce antibodies in the immunized animal that bind to the targeted G17 hormone, thereby inhibiting G17 function and arresting the growth of the G17-dependent tumor cell. The present immunogens induce a biologically effective immune response following administration of the immunogen in all immunized animals tested. The immunomimic peptide-spacer of this invention can be coupled to immunological carriers over a wide range of peptide to carrier substitution ratios and yield effective immunogens.

### Example 1

As shown in U.S. Pat. No. 5,785,970, peptides for the induction of specific immune responses to G17 can, for example, be prepared by standard solid state synthesis methods as follows.

Peptides with the following amino acid sequences were synthesized:

Peptide 1--Human 017 (1-6):
(SEQ ID NO: 1)
pGlu-Gly-Pro-Trp-Leu-Glu-Arg-Pro-Pro-Pro-Pro-Cys
Peptide 2--Human 017 (1-5)
(SEQ ID NO: 2)
pGlu-Gly-Pro-Trp-Leu-Arg-Pro-Pro-Pro-Pro-Cys
Peptide 3--Human G17 (1-4):
(SEQ ID NO: 3)
pGlu-Gly-Pro-Trp-Arg-Pro-Pro-Pro-Pro-Cys
Peptide 4--Human G17 (1-9):
(SEQ ID NO: 4)
pGlu-GlY-Pro-Trp-Leu-Glu-Glu-Glu-Glu-Ser-Ser-Pro-
Pro-Pro-Pro-Cys

Each of the peptides shown consists of an amino-terminal fragment of G17, for example, the first 4-9 amino acids of human G17 in Peptides 1-4, and a carboxy-terminal spacer peptide portion, Arg-Pro-Pro-Pro-Pro-Cys (SEQ ID NO:5), or Ser-Ser-Pro-Pro-Pro-Pro-Cys (SEQ ID NO: 6). Each synthetic peptide was characterized as to amino acid content and purity prior to further preparation of the immunogen.

Each of these peptides was conjugated to amino groups present on a carrier such as Diphtheria toxoid (“DT”) via the terminal peptide cysteine residue utilizing heterobifunctional linking agents containing a succinimidyl ester at one end and maleimide at the other end of the linking agent.

### Example 2

To accomplish the linkage, for example, between any of Peptides 1-4 above and the carrier, the dry peptide was dissolved in 0.1M Sodium Phosphate Buffer, pH 8.0, with a thirty molar excess of dithiothreitol (“DTT”). The solution was stirred under a water saturated nitrogen gas atmosphere for four hours. The peptide containing reduced cysteine was separated from the other components by chromatography over a G10 Sephadex column equilibrated with 0.2M Acetic acid. The peptide was lyophilized and stored under vacuum until used. The carrier was activated by treatment with the heterobifunctional-linking agent e.g. Epsilon-maleimidocaproic acid N-hydroxysuccinimide ester, (“EMCS”), in proportions sufficient to achieve activation of approximately 25 free amino groups per 10⁵ molecular weight of carrier. In the specific instance of diphtheria toxoid, this amounted to the addition of 6.18 mg of EMCS (purity 75%) to each 20 mg of diphtheria toxoid.

Activation of diphtheria toxoid was accomplished by dissolving each 20 mg aliquot of diphtheria toxoid in 1 ml of 0.2M Sodium Phosphate Buffer, pH 6.45. Aliquots of 6.18 mg EMCS were dissolved into 0.2 ml of Dimethyl Formamide (“DMF”). Under darkened conditions, the EMCS was added dropwise in 50 microliter (“μl”) amounts to the DT with stirring. After 2 hours of incubation in darkness, the mixture was chromatographed on a G50 Sephadex column equilibrated with 0.1M Sodium Citrate buffer, pH 6.0, containing 0.1 mM EDTA.

Fractions containing the EMCS activated diphtheria toxoid were concentrated over a PM 10 ultrafiltration membrane under conditions of darkness. The protein content of the concentrate was determined by either the Lowry or Bradford methods. The EMCS content of the carrier was determined by incubation of the activated carrier with cysteine-HCl followed by reaction with 10 mM of Elman's Reagent 5,5′ dithio-bis(2-nitrobenzoic acid) 10 mM. The optical density difference between a blank tube containing cysteine-HCl and the sample tube containing cysteine-HCl and carrier was translated into EMCS group content by using the molar extinction coefficient of 13.6×10³ for 5-thio-2-nitro benzoic acid at 412 nm.

The reduced cysteine content (—SH) of the peptide was also determined utilizing Elman's Reagent. Approximately 1 mg of peptide was dissolved in 1 ml of nitrogen gas saturated water and a 0.1 ml aliquot of this solution was reacted with Elman's Reagent. Utilizing the molar extinction coefficient of 5-thio-2-nitro-benzoic acid (13.6×10³) the free cysteine—SH was calculated. An amount of peptide containing sufficient free—SH to react with each of the 25 EMCS activated amino groups on the carrier was dissolved in 0.1M Sodium Citrate Buffer, pH 6.0, containing 0.1 mM, EDTA., and added dropwise to the EMCS activated carrier under darkened conditions. After all the peptide solution had been added to the carrier, the mixture was incubated overnight in the dark under a water saturated nitrogen gas atmosphere.

The conjugate of the peptide linked to the carrier via EMCS is separated from other components of the mixture by chromatography over a G50 Sephadex column equilibrated with 0.2M Ammonium Bicarbonate. The conjugate eluted in the column void volume is lyophilized and stored desiccated at −20° C. until used.

The conjugate may be characterized as to peptide content by a number of methods known to those skilled in the art including weight gain, amino acid analysis, etc. Conjugates of Peptides 1-3 and diphtheria toxoid produced by these methods were determined to have 20-25 moles of peptide per 10⁵ molecular weight of carrier and all were considered suitable as immunogens for immunization of animals.

### Example 3

An alternative, closed-system method of preparing, conjugating, isolating and purifying peptide-carrier compositions may also be used. Such a method and system are disclosed in U.S. Pat. No. 6,359,114, which is hereby incorporated by reference in its entirety. The method is performed in closed liquid system and consists essentially of the steps of:
(a) conjugating of peptide immunogen with or without spacer to an immunogenic carrier molecule in a liquid reaction mixture, so as to form a mixture of conjugated and unconjugated peptide and other molecules;
(b) ultrafiltering the liquid reaction mixture containing conjugated and unconjugated peptide and other molecules so as to isolate the retentate of conjugated peptide molecules on the ultrafilter of an ultrafiltration means;
(c) washing the isolated retentate of conjugated peptide molecules on the ultrafilter with a desalting solution, water or another buffer solution;
(d) backwashing the ultrafiltration means with a buffer solution from a backwash reservoir to release and disperse the retentate of conjugated peptide molecules from the ultrafiltration means;
(e) purifying the conjugated peptide molecules by repeating the steps (c) and (d) until the conjugated peptide molecules are substantially free of the nonconjugated molecules; and
(f) recovering the retentate of conjugated peptide molecules from the ultrafiltration means, or retransferring the retentate to the reaction vessel from the ultrafiltration means for further modification.

The apparatus is fluidly connected between the reaction vessel and the ultrafiltration/diafiltration device through a suitable fluid pathway such as tubing provided with flow control means such as a valve or pump. The liquid phase of the reaction solution containing reagents and products can be moved from the reaction vessel through a suitable peristaltic pump into the filtration unit. The Diafiltration Reservoir is connected through the reaction vessel to the filtration unit for washing/rinsing of the retentate which is accumulated on the membrane of the filtration unit. The permeate or filtrate can be drained from the filtration unit into the reservoir. The Backflush Reservoir supplies a solution for removing the retentate in a counterflow direction through the ultrafiltration unit into the reaction vessel or other suitable receptacle. Optionally, the fractionation of the protein or peptide containing the reaction products may be sequentially separated into size-graded fractions by using filters with a molecular weight cutoff with an order of magnitude difference in molecular weight or as required to separate the products.

Several combinations of steps and embodiments can be envisioned involving a first purification of at least one of the components involved in a subsequent modification reaction such as conjugation/coupling with one or more other components such as proteins, peptides or nonprotein molecules such as carbohydrates.

Specific part numbers and manufacturers are listed for the various components of the apparatus; however, it is recognized that comparable equipment from other commercial sources may be substituted without diminishing the effectiveness of the apparatus, and it should also be understood that the apparatus can be scaled up to any required level of production without departing from the principles of the invention.

One embodiment of the system may be described in more detail, as follows. The reaction vessel is a 2000 ml, type 1 glass, amber, wide mouth bottle (Wheaton). This vessel was selected based on the following criteria: (i) the 2000 ml capacity accommodates reaction volumes from 100 ml to 1800 ml; (ii) type 1 glass conforms to USP standards for pharmaceutical manufacture; (iii) amber color glass of the reaction vessel limits the penetration of light capable of degrading the light-sensitive chemical crosslinking agent used in the synthesis; and (iv) the wide mouth provides clearance for a stopper fitted with 3 tubes, and it allows easy access for reagent additions and sampling. The wall of the reaction vessel is marked for volume of solution in the vessel, in 100 ml increments. The reaction vessel is capped with a neoprene stopper, which is bored with 3 holes which are equally spaced and located diagonally across the stopper.

Type 1 borosilicate glass tubing of suitable I.D., is passed through each of the 3 holes in the stopper. The reaction vessel is provided with suitable tubing, connected with the pump, and positioned within the vessel so as to effectively evacuate the vessels contents when the pump is in operation.

The exact length of tubing sections is not critical to the operation of the apparatus; however, it is desirable to keep tube lengths as short as practicable to minimize intratube volume. The valves are made of polypropylene and Teflon.

The peristaltic pump is a Model LP1 (Amicon). It is the variable speed, type which allows for adjustment of filter input pressure, and it is reversible.

The Ultrafiltration Unit consists of a spiral membrane cartridge diafiltration concentrator (#54118, Amicon) fitted with a spiral wound membrane cartridge having a suitable molecular weight cut-off. The diafiltration concentrator was selected because its capacity is compatible with the usual reaction volume of the small volume capacity of this embodiment.

The Backwash Reservoir consists of a 500 ml glass separatory (“Buchner”) funnel (#6402. Pyrex) that contains an integral 2-way stopcock vaive.

### Operation 1: Reaction.

Reactions such as for example the chemical conjugation of a short peptide to a larger protein are conducted in the Reaction Vessel. The diafiltration pickup tube 26a is not immersed into the Diafiltration Solution Reservoir 27. Reactants are added to the vessel via opening 101. (Tubing for reagent addition and sample removal tubing can be added to the Reaction Vessel setup, if necessary.) Opening 101 is closed during the reaction period. The reaction mixture is stirred, and the reaction is allowed to proceed to completion. Samples can be withdrawn from the Reaction Vessel to monitor the progress of the reaction.

### Operation 2: Purification.

Purifications are conducted by diafiltration. The Diafiltration Solution Reservoir is filled with diafiltration solution and the glass tubing 26a for diafiltration solution pickup is inserted reaching to the bottom of the Diafiltration Solution Reservoir. The material to be purified is added to the Reaction Vessel, which is then closed. The transfer solution is pumped from the Reaction Vessel through the inlet port into the Ultrafiltration Unit. The Ultrafiltration Unit is operated under the recommended inflow and backpressures by adjusting Pump speed and the Ultrafiltration Unit's integral backpressure valve per the manufacturer's recommendations.

The progress of purification is monitored by testing samples obtained from the tubing leading to the Permeate Reservoir which receives the filtrate drainage of the reaction solution as well as the washing solution. The Diafiltration Solution Reservoir is refilled when low on solution; the Permeate Reservoir is emptied or replaced when appropriate.

When permeate testing indicates that purification is complete the diafiltration solution intake is terminated by for example raising Tubing out of the diafiltrate solution in Diafiltration Solution supply vessel, and the remaining solution is allowed to pass into the Reaction Vessel. Valves therefore are closed. The test solution in the Ultrafiltration Unit and the tubing can then be collected in the Reaction Vessel by draining or backflushing.

The purification operation can also be used to exchange buffers. The same process is followed as for purification, except that the new solvent/buffer is added to the Diafiltration Solution Reservoir. The purification process is allowed to proceed until the old solvent/buffer has been replaced.

### Operation 3: Concentration.

To concentrate solutions in the Reaction Vessel, the appropriate buffer or storage solution is added to the Reaction Vessel. Valve is opened to allow permeate to flow from the Ultrafiltration Unit to the Permeate Reservoir. The diafiltrate uptake tubing is not placed into the Diafiltration Solution Reservoir (to enable air to pass through the tube.) The Pump and the Ultrafiltration Unit are then operated as for the Purification Operation. During the concentration process, the level of solution in the Reaction Vessel must be monitored to ensure that Tubing remains immersed in the solution as the solution level drops. When concentration is complete, the pump is switched off and all Valves are closed. The solution (containing reaction product) in the Ultrafiltration Unit and the tubing can then be drained or backflushed into the Reaction Vessel.

### Operation 4: Draining/Backflushing.

To recover solution containing the reaction product from the Ultrafiltration Unit and the tubing at the conclusion of purification and concentration operations, it is necessary to drain this solution from these components into the Reaction Vessel. To perform this operation step, the diafiltration solution uptake tube is not lowered into the Diafiltration Solution Reservoir, thereby allowing air to pass through the diafiltration tube. Valve is closed. Valve is opened to allow air to pass from the Backwash Reservoir (which is empty) through Valve to Valve. Valve is then opened to allow air to pass from Valve to the Reaction Vessel, thus draining those tubings. To drain the Ultrafiltration Unit, Valve is then adjusted to allow air to pass from Valve to the Ultrafiltration Unit 13. The Pump is activated, in reverse mode, such that the solution with the reaction product flows from the Ultrafiltration Unit through the Pump into the Reaction Vessel. When drainage is complete, the Pump is switched off and Valves closed.

To backflush the Ultrafiltration Unit, the same procedure is followed as for drainage of the Ultrafiltration Unit, except that the desired volume of backwash solution is added to the Backwash Reservoir. Thus, when Valve is opened, only the backwash solution, but not air will flow from the Backwash Reservoir through the Valve into the Ultrafiltration Unit and finally into the Reaction Vessel as receptacle. When backwashing is complete (e.g., the products have been removed), the Pump is switched off and the Valves are closed.

The process of this example is designed for the synthesis of a peptide-protein conjugate that is used for the induction of antibody responses to human gastrin 17 (“hG17”).

This closed process is hereafter explained in more detail as follows:

### Example 4

### Step 1: DT Purification

The DT is provided in a solution that contains other low molecular weight constituents, including 0.3 M glycine and 0.01% thimerosal. These other constituents have to be removed before the conjugation process can begin. The DT is purified by a series of diafiltration and concentration steps using the ultrafiltration unit. Each diafiltration uses a volume of deionized water a diafiltrate solution equal to 5 times the sample volume present in the reaction vessel. To prevent filter clogging, backwash procedures using backflushing from the reservoir are also incorporated into the diafiltration process. Once the diafiltration procedure for DT purification is completed, phosphate buffer (0.5 M sodium phosphate) is substituted using three cycles of diafiltration with 5 fold volumes to prepare for DT activation reaction with EMCS (Epsilon—maleimidocaproic acid N-hydroxysuccinimide ester). At the conclusion of Step 1, the solution is concentrated to about 20-25 mg DT/ml in the ultrafiltration unit (equipped with a spiral wound membrane cartridge of 30,000 MW cut-off; Amicon, YM30S1) by judicious removal of permeate washing solution and by backflushing pure DT into the reaction vessel. DT purity is analyzed by HPLC and the concentration of DT is determined.

### Step 2: Activation of the Purified DT with EMCS

The purified DT is next activated with EMCS, to yield maleimido-DT (MDT). In this step, the succinimidyl moiety of EMCS reacts with free c-amino groups on DT, coupling the EMCS to DT such that the EMCS maleimido group is left to bind peptide (in Step 4).

Of the approximately forty amino groups present per 10⁵ molecular weight of DT protein, about twenty-five are activated in the present synthesis. To achieve this level of activation, a 4-fold molar excess of EMCS to DT amino groups is required. The concentration of DT to be activated is adjusted to 20 mg/ml (+/−0.5 mg/ml) and added back to the reaction vessel. The EMCS is added and maleimido DT (MDT) is formed over a 2 hour reaction period.

### Step 3: Purification of MDT

Non-reacted and hydrolyzed EMCS are next removed from the MDT solution by transferring the reaction mixture from the reaction vessel a series of diafiltration, backwash and concentration steps (as described above) which involve cycling a citrate washing solution from the reaction vessel through the ultrafiltration device, removing the filtrate to reservoir, alternately backwashing from reservoir 22 and concentrating the retained MDT in device, and finally restore the purified MDT to the reaction vessel. In the course of these procedures, citrate (0.1 M sodium citrate) coupling buffer is completely substituted for the phosphate buffer. At the conclusion of this step, the quantity of MDT and its degree of activation are determined.

### Step 4: Conjugation of hG17 Immunogenic Peptide to MDT

The 500 mg of hG17 immunogenic peptide is dissolved in 25 ml of nitrogen gas saturated 0.1 M sodium citrate (SC) and coupled to the activated MDT by gradually adding the purified peptide solution to the purified MDT solution containing 1.17 g MDT at 20 mg/ml 0.1 MSC in the reaction vessel 11 and allowing the coupling reaction to proceed for a suitable time period to completion. Peptide is added at a 1.1:1 molar ratio of peptide:maleimido group (in MDT) to achieve the desired substitution ratio of 25 moles peptide

### Step 5: Conjugate Purification and Lyophilization

The conjugate reaction solution (83.5 ml) was diluted to 1.0 L-volume with 0.2 M ammonium bicarbonate solution (AB) followed by about 5 fold concentration to a volume of approximately 100 mls. This was followed by closed system diafiltration of the solution over a spiral wound membrane of 30,000 Dalton cut-off in the ultrafiltration unit 13 with 500 ml of AB solution effectively retaining only the conjugate and a backwash with 100 ml of AB solution then concentration of the product solution back to 100 ml. This diafiltration-backwash-concentrate process was repeated two more times, followed by 3 cycles of diafiltration-backwash-concentrate process in distilled water. After this final treatment, the system tubing and the membrane cartridge were drained to remove traces of AB. The conjugate solution itself was removed from the reaction vessel and diluted to approximately 2 mg/ml in H₂O and then lyophilized to remove or sublimate any residual AB. The yield of conjugate was found to be 1.4 gm.

The conjugate was analyzed by HPLC and found to contain a single peak indicating homogeneity. By contrast, conjugate produced by the previous methodology was shown by HPLC analysis not to be pure as it contained about three distinct peaks. In addition, the synthesis in this example took only 11/2 days to complete, which is far superior to the 3 days 10 required to perform the synthesis by the previous methodology.

Regardless of the method of conjugation and purification, the immunogenic compositions of the invention may take a variety of forms, for example, solid, semi-solid and liquid dosage forms, such as powders, liquid solutions, suspensions, suppositories, and injectable and infusible solutions. The compositions comprise the present immunogens and suitable pharmaceutically acceptable components, and may include other medicinal agents, carriers, adjuvants, excipients, etc. Suitable adjuvants include, but are not limited to nor-muramyl dipeptide (nor-MDP, Peninsula Labs., CA), and oils such as Montanide ISA 703 (Seppic, Inc., Paris, France), which can be mixed using standard procedures.

In another embodiment of the invention, the method of treatment comprises passive immunization, in which antibodies against G17 are administered to the patient in a sufficient concentration to reduce the levels of circulating unbound G17. The reduced levels of free G17 and G17-Gly in the circulating blood of a patient as a result of anti-G17 antibody administration, results in an inhibition of the growth of the occult or micrometastatic tumor cells. Anti-G17 antibodies for use in passive immunization therapy can, for example, be produced by immunizing a host with the immunogens of Example 1 and thereafter isolating the anti-G17 antibodies from the serum of the host by standard methods such as preparative affinity chromatography. Alternatively, the anti-G 17 antibodies for passive immune therapy may be chimeric, humanized, or human monoclonal antibodies produced by biotechnological methods well known in the art.

The invention also relates to the treatment and/or prevention of cancerous and/or pre-cancerous conditions of the lung, liver, and esophagus by actively and/or passively immunizing a patient against a gastrin receptor, e.g., the CCK-B/gastrin receptor. Immunization against the CCK-B/gastrin receptor may be used alone or in combination with immunization against gastrin itself. Methods for producing immunogens for the production of therapeutic antibodies against the CCK-B/gastrin receptor are disclosed in detail in U.S. application Ser. No. 09/076,372, which is hereby incorporated by reference in its entirety. Antibodies of the invention for passive immunization may be administered to a patient intravenously using a pharmaceutically acceptable carrier, such as a saline solution, for example, phosphate-buffered saline or by any other method.

### Example 5

As shown in U.S. application Ser. No. 09/076,372, an immunogen comprising a peptide from the CCK-B/gastrin-receptor conjugated to an immunogenic carrier molecule can be used to generate an antibody response against the CCK-B/gastrin-receptor in an immunized host. For example, the immunogenic peptide fragment KLNRSVQGTGPGPGASL (SEQ ID NO.: 7 in the Sequence Listing, corresponding to amino acids 5 through 21 of the CCKB/gastrin-receptor sequence) or GPGAHRALSGAPISF (SEQ ID NO.: 8 in the Sequence Listing, corresponding to the fourth extracellular domain of the CCK-B/gastrin receptor) can be used to induce such a response. In one embodiment of the invention, these immunogenic peptides further comprise a carboxy-terminal spacer peptide sequence, such as SSPPPPC (SEQ ID NO.: 6 in the Sequence Listing. The immunogenic carrier can, for example, be selected from the group consisting of Diphtheria toxoid, tetanus toxoid and bovine serum albumin. In one embodiment of the invention the CCK-B/gastrin-receptor immunogenic peptides with spacer are conjugated to the immunogenic carrier Diphtheria toxoid in the same manner as described in Example 1 herein.

An effective dosage of the immunogenic composition includes the range of from 0.001 to 10 mg of the administered to the patient for the treatment of the gastrointestinal cancer. In another embodiment of the invention, adosage of from 0.001 to 2 g is used. The antibody titer levels against the receptor may also be monitored from a sample of blood taken from the patient. Booster immunizations can be given as required to maintain an effective antibody titer.

Anti-CCK-B/gastrin receptor antibodies for passive immunization therapy may also be produced by immunization of a host with the CCK-B/gastrin receptor immunogenic peptide composition, or by any other method known in the art.

The following embodiments of the invention are related in that they all involve actively and/or passively immunizing a patient against G17 gastrin and/or the CCK-B/gastrin receptor.

One embodiment of the invention is directed to the prevention of metastasis of cancer to the liver. For example, a patient at risk of developing a metastatic tumor of the liver, such as a patient with a gastrointestinal malignancy is actively and/or passively immunized against G17 gastrin and/or the CCK-B/gastrin receptor. Another embodiment of the invention is directed to the prevention of metastasis of cancers to the lung. For example, a patient at risk of developing a metastatic tumor of the lung, such as a patient with a gastrointestinal malignancy is actively and/or passively immunized against G17 gastrin and/or the CCK-B/gastrin receptor.

A further embodiment of the invention is directed to the treatment of liver cancer, either originating within the liver itself, or having metastasized to the liver from another site within the body. A similar embodiment of the invention is related to the treatment of lung cancer, either originating within the lung itself, or having metastasized to the lung from another site within the body. Still another embodiment of the invention is directed to the treatment of esophageal cancer, either originating within the esophagus itself, or having metastasized to the esophagus from another site within the body.

In a related embodiment, lung cancer is treated by combined (concomitant or sequential) therapy comprising a taxane, such as docetaxel (Taxotere) or paclitaxel (Taxol), in combination with active and/or passive immunization against G17 gastrin and/or the CCKB/gastrin receptor. According to the invention, in addition to the immunizations against gastrin and/or the gastrin receptor, a dosage of 1-1000 mg/m² of docetaxel or paclitaxel may be administered intravenously once every 3 weeks in the treatment of non-small cell lung cancer. In another embodiment of the invention, lung cancer is treated by combined therapy comprising a platinum compound such as cisplatin, carboplatin or oxaloplatin, in combination with active and/or passive immunization against G17 gastrin and/or the CCK-B/gastrin receptor. The invention also provides these combined therapies for the treatment of liver cancer and esophageal cancer, as well as for the treatment of pre-cancerous conditions of the liver, lung or esophagus. Other chemotherapy agents that may be used singly or multiply in combination with the immunizations of the invention include, but are not limited to, irinotecan, topotecan, 5-fluorouracil plus leucovorin, and gemcitabine.

A further embodiment of the invention is directed to the treatment of the pre-malignant (pre-cancerous) condition, Barrett's esophagus. A related embodiment of the invention is directed to preventing or delaying the progression of Barrett's esophagus to a cancerous state, e.g., adenocarcinoma.

### REFERENCES

1. Watson, S A et al. Antiserum raised against an epitope of the cholecystokinin B/gastrin receptor inhibits hepatic invasion of a human colon tumor. Clin Cancer Res 2000 December; 6(12): 4719-24.
2. Rehfeld, J. F. Three components of gastrin in human serum. Biochim. Biophys. Acta., 285: 364-372, 1972.
3. Upp, J. R, Singh, S., Townsend, C. M., and Thompson, J. C. Clinical significance of gastrin receptors in human colon cancers. Cancer Res., 49: 488-492, 1989.
4. Hoosein, N. M., Kiener, P. A., Curry, R C., and Brattain, M. G. Evidence for autocrine growth stimulation of cultured colon tumor cells by a gastrin/cholecystokinin-like peptide. Exptl. Cell Res., 186: 15-21, 1990.
5. Baldwin, G. S., and Zhang, Q-X. Measurement of gastrin and transforming growth factor a messenger RNA levels in colonic carcinoma cell lines by quantitative polymerase chain reaction. Cancer Res., 52: 2261-2267, 1992.
6. Finley, G. G., Koski, R. A., Melham, M. F., Pipas, J. M., and Meister, A. I. Expression of the gastrin gene in the normal human colon and colorectal adenocarcinoma. Cancer Res., 53: 2919-2926, 1993.
7. Watson, S. A., Durrant, L G., Wencyk, P. M., Watson, A. L, and Morris, D. L. Intracellular gastrin in human gastrointestinal tumor cells. J. N. C. I., 83: 866-872, 1991.
8. Hoosein, N. M., Kiener, P. A., and Curry, R C. Anti-proliferative effects of gastrin receptor antagonists and antibodies to gastrin on human colon carcinoma cell lines. Cancer Res., 48: 7179-7183, 1988.
9. Van-Solinge, W. W., Nielsen, F. C., Friis-Hansen, L., Falkmer, U. G., and Rehfeld, J. F. Expression but incomplete maturation of progastrin in colorectal carcinomas. Gastroenterology, 104: 1099-1107, 1993.
10. Nemeth, J., Taylor, B., Pauwels, S., Varro A., and Dockray, G. J. Identification of progastrin derived peptides in colorectal carcinoma extracts. Gut, 34: 90-95, 1993.
11. Seva, C., Dickinson, C. J., and Yamada, T. Growth-promoting effects of glycine-extended progastrin. Science, 265: 410-412, 1994.
12. Bock, M. G., DiPardio, R M., Evans, R E., Riffle, K. E., Whitter, A., Veber, D, Anderson, E., and Freidinger, A. Benzodiazepine, gastrin and brain cholecystokinin receptor ligands: L-365,260. J. Med. Chem., 32: 13-17, 1989.
13. Hughes, J., Boden, P., Costall, B., Domeney, A., Kelly, E., Norwell, D. C., Hunter, J. C., Pinnock, R. D., and Woodruff, G. N. Development of a class of selective cholecystokinin type B receptor antagonists having potent anxiolytic activity. Proc. Natl. Acad. Sci., 87: 6728-6732, 1990.
14. Watson, S. A., Durrant, L. G., Elston, P., and Morris, D. L. Inhibitory effects of the gastrin receptor antagonist (L-365,260) on gastrointestinal tumor cells. Cancer,
15. Romani, R., Howes, L. G., and Morris, D. L. Potent new family of gastrin receptor antagonists (GRAs) produces in vitro and ill vivo inhibition of human colorectal cancer cell lines. Procs. AACR, 35: 397 (Abstract), 1994.
16. Makishimi, R., Larkin, P., Michaeli, D., and Gaginella, T. S. Active immunization against gastrin-17 with an N-terminal derived immunogen inhibits gastric and duodenal lesions in rats. Gastroent., 106: A824, 1994.
17. Martin, F., Caignard, A., Jeannin, J-F., Leclerc, A., and Martin, M. Selection of trypsin of 2 sublines of rat colon cancer cells forming progressive or regressive tumors. Int. J. Cancer, 32: 623-627, 1983.
18. Ohning, G. V., Wong, H. C., and Walsh, J. H. Differential kinetics for immunoneutralization of circulating gastrin by gastrin monoclonal antibody and its Fab₁ fragment in rats. Peptides, 15: 417-423, 1994.
19. Dickinson, C. J. Relationship of gastrin processing to colon cancer. Gastroenterology, 109: 1384-1388, 1995.
20. Ciccotosto, G. D., McLeish, A., Hardy. K. J., and Shulkes, A. Expression, processing, and secretion of gastrin in patients with colorectal carcinoma. Gastroenterology, 109: 1142-1153, 1995.
21. Kameyama, M., Nakamori, S., Imaoka, S., Yasuda, T., Nakano, H., Ohigashi, H., Hiratsuka, M., Sasaki, Y., Kabuto, T., and Ishikawa, O. “Adjuvant Chemo-endocrine chemotherapy with gastrin antagonist after resection of liver metastasis in colorectal cancer. Gan. To. Kagaku Ryoho (Japan), 21 (13): 2169-2171, 1994.
22. Smith, J. P., Wood, J. G., Solomon, Travis E. Elevated Gastrin Levels in Patients with Colon Cancer or Adenomatous Polyps, 34 (2): 171-174, 1989.
23. Wong, K., Beardshall, K., Water, C. M., Calam, J., G. J. Poston. Postprandial hypergastrinaemia in patients with colorectal cancer, 32: 1352-1354, 1991.
24. Seitz, Jean-Francois, Giovannini, Marc, Gouvemet, Joany, Gauthier, Andre P. Elevated Serum Gastrin Levels in Patients with Colorectal Neoplasia, 13 (5): 541-5, 1991.
25. Goletti et al. Resection of liver gastrinoma leading to persistent Eugastrinemia. Eur. J. Surgery, 158: 55-57, 1992.
26. Katoh et al. Malignant Zollinger-Ellison Syndrome. Stabilizing of liver metastasis after gastrectomy with resection of primary tumor.
27. de Jong et al. Effects of partial liver resection on tumor growth. J. Hepatology, 25: 109-121, 1996.
28. Scheele et al. Indicators of prognosis after hepatic resection for colorectal cancers. Surgery, 110 (1): 13-29, 1991.
29. Vauthey et al. Factors affecting long-term outcome after hepatic resection for Hepatic-cellular carcinoma. The Am. J. Surgery, 169: 28-35, 1995.
30. Hananel et al. Hepatic resection for colorectal liver metastasis. Am. Surgeon 61(5): 444-447, 1995.
31. Slooter et al. Tumor growth stimulation after partial hepatectomy can be reduced by treatment with tumor necrosis factor alpha.
32. Gutman et al. Accelerated growth of human colon cancer cells in nude mice undergoing liver regeneration. Invasion and Metastasis, 14 (1-6): 362-371, 199495.
33. Ballantyne et al. Surgical treatment of liver metastasis in patients with colorectal cancer. Cancer, 71 (12): 4252-4266, 1993.
34. Leith et al. Effects of partial hepatectomy on growth characteristics and hypoxic fractions of xenografted DLD-2 human colon cancers. Radiation Res., 132 (2):263-268, 1992.
35. Matsumata et al. Preliminary report of tumor metastasis during liver regeneration after hepatic resection in rats. Eur. J. Surg. Oncol. 21(2): 188-190, 1995.
36. Vaillant et al. Repeat liver resection for recurrent colorectal metastasis. British J. Surgery 80(3): 340-344, 1993.
37. Mizutani et al. Promotion of hepatic metastases by liver resection in the rat. British J. Cancer 65(6): 794-797, 1992.
38. Ledda-Columbano et al. Compensatory regeneration, mitogen-induced liver growth, and multistage chemical carcinogenesis. Env. Health Persp. 101 (5): 163-168, 1993.
39. Gocyk et al. Helicobacter pylori, gastrin and cyclooxygenase-2 in lung cancer. Med Sci Montior November-December; 6 (6): 1085-1092, 2000.
40. Reddy, A A Small cell lung cancer: improving outcomes. American Society for Therapeutic Radiology and Oncology 42nd Annual Meeting—Day 1, Oct. 22, 2000, meeting report published by Medscape.
41. Evans, T L Chemotherapy in advanced non-small cell lung cancer. 37^{th} Annual Meeting of the American Society of Clinical Oncology—Day 1, May 22, 2001, meeting report published by Medscape.
42. Fennerty, M B Update on Barrett's Esophagus. Digestive Diseases Week 2001 Day 3, May 22, 2001, meeting report published by Medscape.
43. Caplin, M et al. Expression and processing of gastrin In hepatocellular carcinoma, fibromellar carcinoma and cholangiocarcinoma. J Hepatol 1999 March; 30(3): 519-26.

What is claimed is:

1. A method for the treatment of a cancerous condition of the lung or esophagus, comprising:
identifying a patient with a cancerous condition of the lung or esophagus; and
administering to the patient an immunogen that induces antibodies in the patient against gastrin 17 (G17), wherein:
the antibodies inhibit the binding of G17 to its receptor on cancerous cells;
the cancerous condition is gastrin-induced or dependent; and
the cancerous condition of the lung is a primary non-small cell cancerous condition.

2. A method for the treatment of a cancerous condition of the lung or esophagus, comprising:
identifying a patient with a cancerous condition of the lung or esophagus; and
administering to the patient anti-gastrin 17 (G17) antibodies that selectively neutralize the peptide hormone G17 in vivo or inhibit activation of the receptor in vivo,
wherein the cancerous condition of the lung is a non-small cell cancerous condition.

3. A method for the treatment of a cancerous condition of the lung or esophagus, comprising:
identifying a patient with a cancerous condition of the lung or esophagus; and
administering to a patient in need thereof an immunogen that elicits a sufficient titer of antibodies in the patient to effect treatment, wherein:
the elicited antibodies that selectively bind and neutralize the patient's own gastrin 17 (G17);
the cancerous condition is gastrin-induced or dependent; and the cancerous condition of the lung is a non-small cell cancerous condition.

4. The method of claim 2 or claim 3, wherein the condition is a lung cancer, wherein the lung cancer is at least partially gastrin-promoted.

5. The method of claim 4, wherein the lung cancer originated in the lung.

6. The method according to claim 1 or 2, wherein the condition is a cancer of the esophagus.

7. The method of claim 6, wherein the cancer of the esophagus is an adenocarcinoma.

8. The method of claim 1, further comprising an adjuvant chemotherapy comprising administration of cisplatin, carboplatin, oxaloplatin, irinotecan, topotecan, 5-fluorouracil, leucovorin, gemcitabine, and/or a taxane.

9. The method of claim 1, wherein the immunogen comprises an amino acid sequence selected from the group consisting of amino acids 1-4 of human G17, amino acids 1-5 of human G17, amino acids 1-6 of human G17, and amino acids 1-9 of human G17.

10. The method of claim 9, wherein the immunogen further comprises a spacer selected from the group consisting of SEQ ID NOs: 5 and 6 that conjugates the immunogen to a carrier.

11. The method of claim 10, wherein the carrier is selected from the group consisting of Diphtheria toxoid, tetanus toxoid and bovine serum albumin.

12. The method of claim 11, wherein the carrier is Diphtheria toxoid.

13. A method for treating the growth of a gastrin-induced tumor lesion of the lung or esophagus in a patient by selectively neutralizing the peptide hormone gastrin 17 (G17) in vivo, comprising:
identifying a patient with a gastrin-induced tumor lesion of the lung or esophagus; and
administering to the patient anti-G17 antibodies that bind to an epitope located on the amino terminus of G17, thereby inhibiting the binding of G17 to its physiological receptor on the tumor, wherein:
the cancerous condition of the lung is a non-small cell cancerous condition; and
the cancerous condition is gastrin-induced or dependent.

14. A method for the treatment of non-small cell lung cancer, comprising the steps of:
(a) identifying a patient with non-small cell lung cancer;
(b) actively and/or passively immunizing the patient against gastrin, wherein the antibodies provided and/or induced inhibit the binding of gastrin (G17) to its receptor on cancerous cells; and
(c) concomitantly or sequentially administering an effective amount of docetaxel to the patient.

15. A method for the treatment of an esophageal adenocarcinoma in a patient, the method comprising:
(a) identifying a patient with an esophageal adenocarcinoma; and
(b) actively and/or passively immunizing the patient against gastrin, wherein the antibodies provided and/or induced inhibit the binding of gastrin (G17) to its receptor on cancerous cells of the esophageal adenocarcinoma.

16. The method of claim 15, wherein actively and/or passively immunizing step comprises administering to the patient an immunogen comprising an amino acid sequence selected from the group consisting of amino acids 1-4 of human G17, amino acids 1-5 of human G17, amino acids 1-6 of human G17, and amino acids 1-9 of human G17.

17. The method of claim 16, wherein the immunogen further comprises a spacer selected from the group consisting of SEQ ID NOs: 5 and 6 that conjugates the immunogen to a carrier.

18. The method of claim 17, wherein the carrier is selected from the group consisting of Diphtheria toxoid, tetanus toxoid, and bovine serum albumin.

19. The method of claim 18, wherein the carrier is Diphtheria toxoid.
